(19) United States
(12) Patent Application Publication
(10) Pub. No.: US 2011/0189709 A1
(43) Pub. Date: Aug. 4, 2011

(54) METHOD FOR DETECTING HEPATIC INJURY DUE TO ANTITHYROID DRUG

(75) Inventor: Hiroshi MURAYAMA, Chiba-ken (JP)

(73) Assignee: YAMASA CORPORATION, Chiba-ken (JP)

(21) Appl. No.: 13/010,228

(22) Filed: Jan. 20, 2011

(30) Foreign Application Priority Data
Feb. 3, 2010 (JP) 2010-21721

(51) Int. Cl. C12Q 1/52; C12Q 1/48

(52) U.S. Cl. 435/16; 435/15

(57) ABSTRACT

The present invention relates a detecting method of distinguishing a temporary liver function test abnormalities by a thyrotoxicosis from a drug-induced hepatic injury by an antithyroid drug using a blood test and the likes, which is performed in a simple manner and with specificity. In the course of treating Graves' disease using an antithyroid drug, it is possible to differentiate a temporary liver function test abnormalities resulted from a thyrotoxicosis from a drug-induced hepatic injury resulted from the antithyroid drug, and therefore to detect the drug-induced hepatic injury with specificity by assaying a level of OCT in a body sample from a patient. In other words, it is possible to diagnose as a drug-induced hepatic injury if a blood OCT level in the sample from the patient is elevated compared to a level prior to administering the antithyroid drug or a blood OCT level from normal persons. Meanwhile, if the OCT level is not changed in spite of elevated levels of transaminases in the sample, it is possible to continue the treatment without discontinuing the drug administration since liver function test abnormalities in this case can be judged as temporary abnormalities owing to metabolic alteration, not as a drug-induced hepatic injury.

This application claims the benefit of Japanese Patent Application No. 2010-21721, filed on Feb. 3, 2010, which is hereby incorporated by reference in its entirety.

### BACKGROUND OF THE INVENTION

1. Field of the Invention

The present invention relates to a method for detecting a hepatic injury, and more particularly, to a method of distinguishing between a temporary liver function test abnormalities (hepatic malfunction) owing to a thyroid hormone-induced toxicosis (thyrotoxicosis) and a drug-induced hepatic injury owing to an antithyroid drug by measuring a level of ornithine carbamyltransferase (OCT) in a body sample from a patient.

2. Description of the Related Art

It is frequently observed that a liver function test abnormalities such as elevated levels of blood aspartate transaminase (AST) or alanine transaminase (ALT) in patients suffering from thyroid diseases such as Graves' disease, painless thyroiditis, acute thyroiditis. Typically, it has been thought that a thyrotoxicosis resulting from excessively high concentration level of a thyroid hormone in blood causes the liver function test abnormalities. In addition, since it has been reported that an antithyroid drug administered to patients suffering from Graves' disease causes a hepatic injury as a side effect, the antithyroid drug is regarded as a candidate causing the liver function test abnormalities in the thyroid disease (see non-patent document 1, which is incorporated herein by reference).

Accordingly, when the temporary liver function test abnormalities is observed from patients commencing a treatment using an antithyroid drug, it has been thought that the abnormalities are caused either by the side effect of the antithyroid drug or by a toxicosis owing to a thyroid hormone. Particularly, since the drug-induced hepatic injury owing to the side effect of the antithyroid drugs has been relatively frequently developed, and the patient could die in the worst case, it is a common sense that an administration of the drug is discontinued so as to avoid the side effect when the transaminase levels are highly elevated.

Recently, it has been reported that some liver function test abnormalities after treatment with an antithyroid drug are temporary phenomenon owing to metabolic alteration (see non-patent document 2, which is incorporated herein by reference). This document guesses that treatment using the antithyroid drug causes some degrees of metabolic alteration and induction of enzymes such as AST and ALT, thereby blood enzyme levels are elevated. However, the document does not certify whether or not a hepatic injury is actually occurred by the elevated level of blood thyroid hormone and whether or not enzymes such as AST and ALT are actually induced in the liver.

As a result, the document (see non-patent document 3, which is incorporated herein by reference) just describes that treatment using the antithyroid drug should be continued and the patient's conditions should be followed carefully if the level of ALT is less than 150 IU/L even when the level of blood ALT is elevated in the course of the treatment using the antithyroid drug. In addition, the document does not describe a hepatic injury marker that can be effectively used in diagnosing whether the liver function test abnormalities such as an elevation of ALT level are caused by temporary thyrotoxicosis or by a drug-induced hepatic injury.

Furthermore, it has been recommended to check for jaundice or to measure a level of blood bilirubin in addition to the fluctuation of transaminases such as ALT for detecting the liver function test abnormalities resulting from the drug-induced hepatic injury (see non-patent document 3). However, this document does not concretely evaluate whether or not bilirubin enables the medical practitioners to detect the drug-induced hepatic injury more sensitively as compared with ALT.

Moreover, it has been known that the drug-induced hepatic injury may be classified into a cholestatic type, a hepatocellular injury type and a mixed type. Among those types, the drug-induced hepatic injury owing to a side effect by an antithyroid drug of propylthiouracil (PTU) usually has the hepatocellular injury type or the mixed type. However, it has been well known that the measurement of bilirubin is useful only for detecting the cholestatic type hepatic injury or the mixed type hepatic injury and is not useful for detecting the hepatocellular injury type. Accordingly, it is difficult to detect the drug-induced hepatic injury owing to the side effect of PTU by the bilirubin, and therefore, the bilirubin is not proper for detecting the hepatic injury owing to the side effect of the antithyroid drug clinically.

Under those typical circumstances, when the liver function test abnormalities such as the blood ALT level over 150 IU/L are observed in the course of the treatment with the antithyroid drug, the drug administration should be discontinued because it cannot be certified whether the liver function test abnormalities are caused by a drug-induced hepatic injury or a temporary change in metabolism resulting from a thyrotoxicosis. Further, in the conventional diagnostic standard, even when the elevation of ALT level is observed in administering the antithyroid drug, the elevated level of ALT may not be diagnosed as a hepatic injury by the side effect of the antithyroid drug if the elevation degree of ALT is relatively low. In this case, the patient's life may be risked because adequate treatments such as discontinuation of the drug treatment are not performed.
Patent document 1: WO 2007/122799
Non-patent document 1: Ann Intern Med 118(6): 424-8, 1993
Non-patent document 2: Thyroid 18(3): 283-7, 2008
Non-patent document 3: The 51^{st} Japanese Thyroid Society Expert Medical Education Seminar II
Non-patent document 4: Clinica Chimica Acta 375:63-58, 2007
Non-patent document 5: Clinica Chimica Acta 391: 31-35, 2008

According to conventional knowledge, although it is certified that the thyrotoxicosis causes the elevation of blood ALT and AST levels, it is not apparent that the hepatic injury is actually caused when the blood ALT and AST levels are elevated (see non-patent documents 2 and 3).

In addition, even if it is certified that the level of blood AST or ALT is elevated by the thyrotoxicosis, the liver function test abnormalities will be relieved by lowering a blood hormone level due to continuous administration of the antithyroid drug. However, if the drug-induced hepatic injury is caused by the side effect of the antithyroid drug, the continuous administration of the antithyroid drug may cause a severe liver injury and risk a patient's life.

Therefore, when the liver function test abnormalities are observed from the patients in the treatment with the administration of the antithyroid drug, it is necessary to select correctly one medical treatment between: 1) the verification that the liver function test abnormalities are temporarily caused by the thyrotoxicosis and the continuous administration of an antithyroid drug to lower the blood hormone level irrespective of a hepatic injury or 2) the verification that the liver function test abnormalities are caused by side effect of an antithyroid drug and the prompt discontinuation of the drug administration.

Therefore, in the course of the treatment with the antithyroid drug, it is extremely important clinically to distinguish between the temporary elevated levels of transaminases owing to thyrotoxicosis, that is, the temporary liver function test abnormalities and the drug-induced hepatic injury resulting from the antithyroid drug. However, there has not been a method for specifically detecting the hepatic injury resulting from the antithyroid drug in a simple manner such as a blood test or a urine test.

Meanwhile, the inventor and the co-researchers have found that ornithine carbamyltransferase (OCT) leaks earlier than transaminase such as AST and ALT in animal models for a drug-induced hepatic injury. For example, it has been reported that blood OCT is leaked earlier than ALT and AST when an liver injury are caused in a rat by administering thioacetamide (see non-patent document 4, which is incorporated herein by reference, and patent document 1) or when a hepatic injury is induced in a rat by administering various substances such as carbon tetrachloride, allyl alcohol, D-galactosamine, lipopolysaccharide, and concanavalin A (see non-patent document 5, which is incorporated herein by reference).

It has never been reported if the blood OCT level is elevated in both cases of the thyrotoxicosis and the drug-induced hepatic injury owing to the antithyroid drug. On the contrary, it has been known that OCT is leaked into blood in extremely various hepatic injury models, and therefore, OCT is a sensitive liver injury marker. Therefore, it has been supposed by a skilled person that OCT is not useful as a maker for distinguishing the cause of the hepatic injury because the blood OCT level is elevated irrespective of the hepatic injury caused by either the thyrotoxicosis or the side effect owing to the antithyroid drug. Moreover, even if the hepatic injury is not caused by the thyrotoxicosis, it cannot be expected that OCT is a marker for distinguishing between the hepatic injury caused by the thyrotoxicosis and the side effect resulting from the antithyroid drug, since the fluctuation of blood OCT level in patients suffering from thyrotoxicosis has not been reported so far.

As such, the skilled person has expected that it is difficult to efficiently distinguish between the temporary liver function test abnormalities owing to the thyrotoxicosis and the drug-induced hepatic injury owing to the antithyroid drug by measuring the blood OCT level in patients in the course of the treatment with the antithyroid drug. Accordingly, it has never been considered to use OCT as a marker of antithyroid drug-induced hepatic injury.

### BRIEF SUMMARY OF THE INVENTION

Accordingly, the present invention is directed to a method for detecting a hepatic injury due to an antithyroid drug that substantially obviates one or more of the problems due to limitations and disadvantages of the related art.

An object of the present invention is to provide a method for detecting antithyroid drug-induced hepatic injury by assaying the OCT level in a body sample.

Another object of the present invention is to provide to a method for diagnosing a temporary hepatic injury or metabolic alteration where an administration of an antithyroid drug can be continued.

To achieve these and other advantages and in accordance with the purpose of the present invention, as embodied and broadly described, a method for detecting a hepatic injury due to an antithyroid drug includes assaying an elevated level of OCT (Ornithine carbamyltransferase) in a body sample after administering an antithyroid drug compared to a level of OCT prior to administering the antithyroid drug.

Also, the present invention provides a method for detecting a hepatic injury owing to an antithyroid drug, the method comprises the steps of: assaying a body sample for an elevated level of OCT (Ornithine carbamyltransferase) after administering an antithyroid drug; and correlating the elevated level of OCT after administering the antithyroid drug compared to a level of OCT prior to administering the antithyroid drug.

Besides, the present invention provides a method for detecting a hepatic injury by an antithyroid drug, the method comprising measuring an elevated level of OCT (Ornithine carbamyltransferase) in a body sample from a subject under treatment by an antithyroid drug compared to a level of OCT in a sample from healthy person.

Further, the present invention provides a method for detecting a hepatic injury by an antithyroid drug, the method comprises the steps of: measuring an elevated level of OCT (Ornithine carbamyltransferase) in a body sample from a subject under treatment; and correlating the elevated level of OCT in a body sample from a subject with a hepatic injury by the antithyroid drug compared to a level of OCT in a body sample from healthy persons.

Still further, the present invention provides a method for detecting a hepatic injury, the method comprises the steps of: measuring levels of transaminase and OCT (Ornithine carbamyltransferase) in a body sample from a subject under treatment of an antithyroid drug; judging as a drug-induced hepatic injury in case the levels of transaminase and OCT are elevated compared to levels of transaminase and OCT prior to administering the antithyroid drug; and judging as a thyrotoxicosis in case the level of the transaminase is elevated and the level of OCT is not changed compared to levels of transaminase and OCT prior to administering the antithyroid drug.

Using the present method, it is possible to discern if the liver function test abnormalities such as elevated levels of blood transaminases in the course of antithyroid drug treatment is caused temporarily by thyrotoxicosis or caused by a drug-induced hepatic injury owing to side effects of an antithyroid drug. Accordingly, it is possible to discern a temporary liver function test abnormalities owing to an thyrotoxicosis from a drug-induced hepatic injury owing to an antithyroid drug and therefore, detect the drug-induced hepatic injury specifically by observing OCT level in a body sample such as blood obtained from a patient in the course of treatment of thyroid diseases such as Graves' disease using the antithyroid drug.

In other words, if the OCT level in a body sample obtained from a patient is elevated compared to an OCT level prior to administering an antithyroid drug or an OCT level in a body sample obtained from normal persons, it is possible to know the development of the drug-induced hepatic injury. On the contrary, if the OCT level is not changed in spite of the elevated levels of transaminase in the sample, it is possible to continue to treat using the drugs without discontinuing administration of drugs meaninglessly, because it is possible to know that the liver function test abnormalities are not caused by a drug-induced hepatic injury, but caused temporarily by metabolic alteration in this case.

As such, it is possible to detect the drug-induced hepatic injury resulted from the antithyroid drugs early and specifically by measuring OCT levels in a sample obtained from a patient in the course of antithyroid drug treatment. Besides, since it is possible to detect the drug-induced hepatic injury only by measuring OCT levels without measuring levels of transaminases in the sample, the present invention is clinically useful.

Additional features and advantages of the invention will be set forth in the description which follows, and in part will be apparent from the description, or may be learned by practice of the invention. The objectives and other advantages of the invention will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

FIG. 1 is a view illustrating each of rates of leaked OCT, ALT and AST from primary cultured hepatocytes treated with PTU. The horizontal axis defines hours after PTU addition and the vertical axis defines the percentage of leaked substances (markers).

### DETAILED DESCRIPTION OF THE INVENTION

To solve the problems of the related art, the inventor of the present invention has done research repeatedly and found that OCT level is not changed while the blood transaminase levels are elevated in a rat model of thyrotoxicosis, which is contrary to the conventional expectations and knowledge, although OCT is leaked predominantly compared to transaminases, which is alike the related art, in case a high concentration of an antithyroid drug is added to a primary cultured hepatocyte of rat.

An “antithyroid drug” herein is not limited to a particular drug if it is generally used, for example, includes thiamazole (mercazole, MMI), propacil (propylthiouracil, PTU) and the likes.

A subject sample for assaying is not limited to a particular one if the sample is originated from a blood, such as a blood, a serum and a blood plasma. Those samples are obtained from patients treated for Graves' disease using the antithyroid drug.

It is possible to use known processes for measuring OCT in the sample, for example, generally accepted technique such as enzymatic method, ELISA (Enzyme-linked immunosorbent assay), RIA (Radioimmunoassay), Chemiluminescence method, Latex agglutination, and other process can be used. With regard to each process, it is possible to adopt known procedures and conditions.

The detection between the temporary liver function test abnormalities owing to the thyrotoxicosis and the drug-induced hepatic injury owing to the antithyroid drug is performed as indicated below. In the detection method, the OCT level before administering the antithyroid drug is compared with the OCT level after administering the antithyroid drug. When the OCT level after administering the antithyroid drug is elevated as compared with the OCT level before administering the antithyroid drug, it is judged with high probability that a drug-induced hepatic injury has developed. When the OCT level in a sample obtained from a subject who is treated with an antithyroid drug is higher than the OCT level in a sample obtained from a normal person, it is judged with high probability that a hepatic injury owing to an antithyroid drug has developed. It is possible to select one of these two ways. Specifically, when a drug-induced hepatic injury is speculated based upon the above-mentioned method, it is preferable to discontinue administering the antithyroid drug.

In addition, it is also possible to refer to the other marker values such as ALT and AST in discerning the drug-induced hepatic injury from the temporary thyrotoxicosis. For example, if the OCT level does not change after administering the antithyroid drug as compared with that before administering the antithyroid drug, it is possible to diagnose that liver function test abnormalities are caused by a temporary thyrotoxicosis so as to continue to administer the antithyroid drug, even when the level of ALT in a sample is elevated.

The present invention will be described in detail by way of examples, which should not be construed as limiting the present invention.

### Example 1

### Rat Thyrotoxicosis Model

Ten male Wistar rats were divided into two groups: “a T3-administration group” and “a control group”. A thyroid hormone (T3) (0.1 mg/kg/day) was administered subcutaneously to the T3-administration group, while a physiological saline solution was administered subcutaneously to the control group, for 10 days. Each of blood OCT, AST and ALT levels was measured before and after the administration. Subsequently, the liver was sampled and homogenized in phosphate buffer for the measurement of each enzyme level in the liver. The OCT level was assayed by ELISA (see non-patent document 3), and the ALT and AST levels were assayed by using a commercially available Kit (Transaminase C-II Test Wako, Wako Pure Chemical Industries, Ltd. Japan).

The weight of T3-administration group was significantly suppressed by administering T3 for 10 days as compared with the control group (T3-administration group 266±5.8 g; Control group 303±15.9 g). Since it is well known that body-weight is lost owing to hyper-metabolism in case of excess thyroid hormone, it is certified that the thyroid hormone T3 administered to the T3-administration group was functioning.

Moreover, the T3-administration group showed significant elevations in blood ALT level (T3-administration group 11.2±0.6 IU/L; control group 8.2±0.8 IU/L) and blood AST level (T3-administration group 40.0±6.51 IU/L; control group 29.0±5.1 IU/L) compared to the control group. On the contrary, there was no significant variation in OCT level among the groups (T3-administration group 11.9±2.5 ng/mL; control group 14.0±1.6 ng/mL).

Similarly to the blood levels, each enzyme level in the liver was assayed. In the T3-administration group, the ALT and AST contents in the liver were elevated significantly while the OCT content in the liver tissue did not change. The blood ALT and AST level correlated positively with the ALT and AST content in the liver, that is, the higher the blood ALT or AST level, the higher the ALT or AST content in the liver. However, there was no such a relationship between the OCT content in the liver and the blood OCT level.

Accordingly, it is suggested that changes in metabolism owing to T3 administration have an effect on the contents of AST and ALT in the liver so as to induce changes in blood levels of AST and ALT.

### Example 2

### Antithyroid Drug (PTU or MMI)-Induced Hepatotoxicity Model in Rats

Eighteen male Wistar rats were divided into three groups: “a PTU-administration group,” “an MMI-administration group” and “a control group.” 250 mg/kg/day of PTU, 200 mg/kg/day of MMI and a physiological saline solution were administered orally to the PTU-administration group, the MMI-administration group and the control group, respectively, for 7 days. Seven days after the start of administration, blood OCT, AST and ALT levels were measured. Subsequently, the liver was sampled and homogenized in phosphate buffer for the measurement of each enzyme level in the liver. The OCT level was assayed by ELISA (see non-patent document 3) and the ALT and AST levels were assayed by using a commercially available Kit (Transaminase C-II Test Wako, Wako Pure Chemical Industries, Ltd. Japan).

While the PTU-administration group showed the significantly elevated blood ALT level, the MMI-administration group showed the lowered ALT level, compared to the control group (PTU-administration group 9.5±0.5 IU/L; MMI-administration group 6.2±1.7 IU/L; control group 7.2±0.6 IU/L). Similarly to the ALT blood level, the AST level was elevated significantly in the PTU-administration group and lowered significantly in the MMI-administration group, compared to the control group (PTU-administration group 31.9±6.0 IU/L; MMI-administration group 9.8±2.4 IU/L; control group 24.7±0.8 IU/L). On the other hand, the OCT level was elevated significantly in both the PTU-administration group and the MMI-administration group compared to the control group (PTU-administration group 37.5±4.4 ng/mL, MMI-administration group 40.0±14.2 ng/mL, control group 16.2±1.4 ng/mL). The degree of elevation of OCT in the drug-administration groups was 2-3 times higher than that of the control group, which was larger than that of ALT and AST (about 1.3 times) in the PTU-administration group.

As indicated above, an ALT content in the liver of the MMI-administration group was decreased significantly compared to the control group. Moreover, an AST content in the liver of the MMI and PTU-administration group was decreased significantly compared to the control group. None of the groups showed changes in an OCT content in the liver.

The liver was histologically evaluated in order to certify that administration of the antithyroid drug caused an actual hepatic injury. As a consequence of the histological evaluation, mild to moderate fatty degeneration was observed in over 90% hepatocytes of PTU-administration group. The MMI-administration group showed moderate cloudy swelling and focal necrosis in addition to the fatty degeneration. From the result of the histological evaluation, it is apparent that administrations of the antithyroid drug caused at least some damages to hepatocytes, and therefore, it is confirmed that this antithyroid drug administration model was proper for the evaluation of drug-induced hepatic injury.

From the above results, it is apparent that the OCT level is elevated significantly, while the level of ALT or AST is slightly elevated, is not elevated at all, or even decreased in the hepatic injury caused by the administration of the antithyroid drug.

### Example 3

### OCT Leakages Into Culture Medium In hepatotoxicity Model Using Primary Cultured Hepatocytes

Rat hepatocytes, which were isolated by collagenase prefusion, are placed into a 24-well plate coated with collagen to have 10⁵ cells/well and cultured for 4 hours. After removing the cells not fixed to the well, 10 mM of PTU was added to the cells and each of the 3 cultured media prior to adding the PTU, of 1 hour after adding the PTU, and of 3 hours after adding PTU, was sampled. After 3 hours of culturing, 0.5% of Triton-X100 was added to destroy the cells and a suspended solution was obtained. OCT, ALT and AST levels in each of the sampled cultured medium and the suspended solution after cell destruction were measured. The OCT level was assayed by ELISA, and the ALT and AST levels were assayed by using a commercially available Kit. Based upon an activity of the suspended solution as 100%, leakage rates of OCT, AST and ALT were calculated in each cultured medium obtained by sampling.

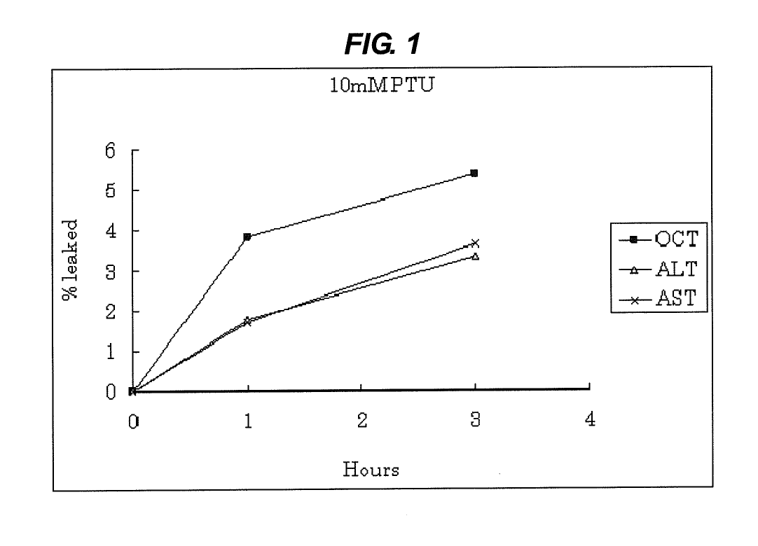

As a result, all of the levels of AST, ALT and OCT, which are liver proteins, were elevated by adding PTU (see FIG. 1). In addition, since the OCT leakage rates for 1 hour and 3 hours after adding the drug were higher compared to the ALT leakage rates, it is shown that OCT is useful comparing to transaminase such as AST or ALT in early detection of cell injury by PTU.

From the results in examples 1-3, it is apparent that thyroid hormone or the antithyroid drug affects on the ALT and AST contents in the liver and changes in the ALT and AST contents in the liver affect on the blood ALT and AST level. Furthermore, the OCT content in the liver is not affected by the thyroid hormone or the antithyroid drug. From these results, it is certified that blood OCT level is superior for the detection of a hepatic injury owing to the antithyroid drug and it is not proper to use the blood AST and ALT levels for detecting the hepatic injury induced by antithyroid drugs.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

What is claimed is:

1. A method for detecting a hepatic injury due to an antithyroid drug, the method comprising: assaying an elevated level of OCT (Ornithine carbamyltransferase) in a body sample after administering an antithyroid drug compared to a level of OCT prior to administering the antithyroid drug.

2. The method of claim 1, wherein the body sample includes a sample from patients treated for Graves' disease using the antithyroid drug.

3. A method for detecting a hepatic injury due to an antithyroid drug, the method comprising:
assaying a body sample for an elevated level of OCT (Ornithine carbamyltransferase) after administering an antithyroid drug; and
correlating the elevated level of OCT after administering the antithyroid drug compared to a level of OCT prior to administering the antithyroid drug.

4. The method of claim 3, wherein the body sample includes a sample from patients treated for Graves' disease using the antithyroid drug.

5. A method for detecting a hepatic injury due to an antithyroid drug, the method comprising: measuring an elevated level of OCT (Ornithine carbamyltransferase) in a body sample from a subject under a treatment using an antithyroid drug compared to a level of OCT in a sample from a healthy person.

6. The method of claim 5, wherein the body sample includes a sample from patients treated for Graves' disease using the antithyroid drug.

7. A method for detecting a hepatic injury due to an antithyroid drug, the method comprising:
measuring an elevated level of OCT (Ornithine carbamyltransferase) in a body sample from a subject under treatment; and
correlating the elevated level of OCT in the body sample from the subject with a hepatic injury due to the antithyroid drug compared to a level of OCT in a sample from a healthy person.

8. The method of claim 7, wherein the body sample includes a sample from patients treated for Graves' disease using the antithyroid drug.

9. A method for detecting a hepatic injury, the method comprising:
measuring levels of transaminase and OCT (Ornithine carbamyltransferase) in a body sample from a subject under treatment using an antithyroid drug;
judging as a drug-induced hepatic injury when the levels of transaminase and OCT are elevated as compared with levels of transaminase and OCT prior to the treatment using the antithyroid drug; and
judging as a thyrotoxicosis when the level of transaminase is elevated as compared with the level of transaminase prior to the treatment using the antithyroid drug and the level of OCT is substantially the same as the level of OCT prior to the treatment using the antithyroid drug.

10. The method of claim 9, wherein the body sample includes a sample from patients treated for Graves' disease using the antithyroid drug.
